(19) United States
(12) Patent Application Publication
(10) Pub. No.: US 2006/0089410 A1
(43) Pub. Date: Apr. 27, 2006

(54) Combination of chemotherapeutic drugs for increasing antitumor activity

(75) Inventors: Louis R Bucalo, South San Francisco, CA (US); Sunil Sreedharan, South San Francisco, CA (US)

Correspondence Address: HELLER EHRMAN LLP, 275 MIDDLEFIELD ROAD, MENLO PARK, CA, 94025-3506, US

(73) Assignee: Titan Pharmaceuticals, Inc., South San Francisco, CA (US)

(21) Appl. No.: 10/521,548

(22) PCT Filed: Jul. 11, 2003
(86) PCT No.: PCT/US03/22181

§ 371 Date: Aug. 22, 2005

Related U.S. Application Data
(60) Provisional application No. 60/396,667, filed on Jul. 17, 2002.

(51) Int. Cl. A61K 31/7072; A61K 31/513; A61K 31/66; A61K 31/33; A61K 33/24; A61K 31/28; A61K 31/22

(52) U.S. Cl. 514/546; 514/492; 514/49; 514/269; 514/109; 514/183; 424/649

(57) ABSTRACT

An oxyalkylene group containing histone deacetylase(s) inhibitor is used sequentially with another antineoplastic agent to increase antitumor activity in cells and mammals.

BACKGROUND OF THE INVENTION

1. Field of the Invention

The present invention relates to the biologically active agent pivaloyloxymethyl butyrate, known commercially as PIVANEX, used in combination with chemotherapeutic agents, pharmaceutical compositions containing them, and methods for treating mammals. The invention demonstrates that the combination of an oxyalkylene containing compound and chemotherapeutic agents increases antitumor activities for a number of different cancer cells.

2. Description of Related Art

Butyric acid is a non-toxic natural product found in butter in concentrations of up to approximately 5%. In the digestive system it is secreted as a product of microbial fermentation. In the colon it can reach mM concentrations. It is known that butyric acid, whether in free form or more usually in the form of its alkali metal salts (“butyric acid/salts”), displays antineoplastic activity. In particular, this activity is evidenced in the form of toxicity towards neoplastic cells, inhibition of cell proliferation, and induction of cytodifferentiation. Such activity has been demonstrated both in vitro and in vivo.

Thus, e.g. in a variety of tumor cells grown in vitro, there has been reported anti-tumor activity of butyric acid/salts due to the induction of morphological and biochemical changes. Some representative examples of affected cells derived from human sources are: neuroblastoma [Prasad and Kumar, Cancer 36:1338 (1975)]: leukemia [Collins et al. Proc. Natl. Acad. Sci. 75:2458 (1978)]; colon carcinoma [Dexter et al. Histochem. 16:137 (1984)] and Augeron and Laboisse, Cancer Res. 44:3961 (1984)]; pancreatic carcinoma [McIntyres et al, Euro. J. Cancer Clin. Onc. 20:265 (1984)]; kidney tumor cells [Heifetz et al. J. Biol. Chem. 256:6529 (1981)]; breast cancer [Stevens et al, Biochem. Biophys. Res. Comm. 119:132 (1984)]; prostatic carcinoma [Reese et al, Cancer Res. 45:2308(1985)]; astrocytoma [McIntyre, J. Cell. Sci. 11:634 (1971)]; human epidermoid carcinoma [Marcher et al, Exp. Cell. Res. 117:95 (1978)]. Moreover, in all in vitro tests carried out by the present inventors, on leukemic cells isolated from myelogenous leukemic patients, butyric acid/salts was found to be the most potent cytotoxic and cytodifferentiating agent, being for example, more effective than retinoic acid, 1, 25-dihydroxy vitamin D and cytosine arabinoside.

Reported examples of in vivo application of butyric acid/salts are as follows. Patients with neuroblastoma received doses of up to 10 g/day, which produced no clinically detectable toxicity [Prasad, Life Sci. 27:1351 (1980)]. Treatment of a child with refractory acute myelogenous leukemia in relapse, with 0.5 g/kg/day, resulted in partial and temporary remission without detectable toxic effects [Novogrodsky et al. Cancer 51:9 (1983)]. Furthermore, the present inventors have treated a patient with acute myelogenous leukemia in relapse, with 1.0 g/kg/day for 10 days and 1.5 g/kg/day for an additional 6 days; the clinical follow up showed no adverse reaction [Rephaeli et al, Blood 68:192a (1986)]. Clinical trials with high dosages of butyric acid/salts resulted in no toxicity.

Tie selectivity of butyric acid/salts was demonstrated, in hitherto unpublished work (by M. Shaklai and E. Januszewiez) by inhibition of colony forming units, granulocytes and macrophages (CFU-GM), grown in soft agar, obtained from normal bone marrow and from peripheral blood of leukemic patients.

Suzanne M. Cutts et al., Cancer Research 61, 8194-8202 described the synergistic interaction of doxorubicin (Adriamycin®) with pivaloyloxymethyl butyrate when both drugs are exposed simultaneously to cells or when pivaloyloxymethyl butyrate is up to 18 hours after doxorubicin administration. The authors report that the reverse order of addition results in antagonism.

Elena di Gennaro et al., Abstract Number: 3636 in the Proceedings of the AACR, Volume 44, March 2003 indicate that the 24 hour pretreatment with the histone deacetylase inhibitor SAHA followed by raltitrexed or 5FU produced a potentiation of the synergistic interaction. SAHA is suberoylanilide hydroxamic acid.
A. Patnaik et al., Clinical Cancer Research Vol.8, 2142-2148, July 2002 report that combinations of AN-9 (pivaloyloxymethyl butyrate) and docetaxel, gemcitabine or cisplatin used in vitro have more than additive cytotoxic effects against a variety of cells lines, but do not discuss the order of the addition of the chemotherapeutic agents.

### SUMMARY OF THE INVENTION

The present invention provides a method of treating cancer, in particular, treating cancers in mammals, comprising administering sequentially a therapeutically effective amount of a composition comprising an oxyalkylene containing histone deacetylases inhibitor (HDAC), followed by the administration of other chemotherapeutic agents. In one embodiment of the invention, the oxyalkylene containing HDAC inhibitor is pivaloyloxymethyl butyrate. Surprisingly, and in contrast to the experience reported doxorubicin, it has been found that this order of addition results in more than additive efficacy.

We have discovered a more than additive inhibition of the growth of cancer or other tumors in humans or animals that occurs in the course of sequential administration of a therapeutically effective amount of an oxyalkylene containing compound, followed by the administration of certain other chemotherapeutic agents, and/or optionally other cancer treatments to the site of the cancer. We have also observed antagonistic (anti-additive) effects, if the sequence of administration is being reversed. The enhanced inhibition is particularly pronounced at higher doses of the oxyalkylene containing compound, in particular at doses between more than 2, in particular about 3, 4, 5, 6 or 7 g/m²/day of Pivanex in mammals or at concentrations of more than 125 μM, that is more than 150, 160, 170, 180, 190, 200, 210, 220, 230, 240, or 250 μM of Pivanex in tumor cells. These increased dosages or concentrations are designed to achieve a substantial reduction of the need to Pivanex exposure, or of the Pivanex induction period (described and defined in more detail herein), to sensitize tumor cells to the effect of chemotherapeutic agents. Applying these increased dosages or concentrations would permit reduction of the Pivanex induction period to less than 60, 48, 36, 24, 12, 6, 4, and slightly more than 2 hours.

More specifically, this invention provides an anti-cancer method of treatment comprising a pharmaceutical carrier and an oxyalkylene containing compound, followed sequentially by the treatment with other chemotherapeutic agents, and/or optionally, other cancer treatments as defined herein along with a method for treating such cancers.

The invention also concerns the use of a HDAC inhibitor in the manufacture of a chemotherapeutic preparation for increasing the antitumor activity of said HDAC inhibitor which includes the use of a chemotherapeutic agent of the class consisting of tubulin interactors, DNA-interactive agents, DNA-alkylating agents, and platinum complexes in said manufacture, said preparation being adapted for an induction period during which the HDAC inhibitor is administered, followed by administration of said chemotherapeutic agent.

The pharmaceutical compositions of the invention may be adapted for oral, parenteral or rectal, topical and other modes of administration including intraurethral, intravaginal, intrabladder, etc. administration, and may be in unit dosage form, as is well known to those skilled in the pharmaceutical art.

The invention further relates to a method of treating tumors or producing an immune response modulating effect in animals, which comprises sequential administration to a mammal of an effective antitumorogenic or immune response modulating dose of an oxyalkylene containing compound, followed by the administration of one or more chemotherapeutic agents, and/or optionally other cancer treatments to the site of the cancer. The invention also includes the use of a compound comprising an oxyalkylene containing compound in conjunction with other chemotherapeutic agents for the manufacture of a medicament for treating tumors or for producing an immune modulating response in animals. The compounds described herein will be effective in both human and non-human animals.

Additional objects, features and advantages of the invention will be set forth in the description which follows, and in part, will be obvious from the description, or may be learned by practice of the invention. The objects, features and advantages of the invention may be realized and obtained by means of the instrumentalities and combination particularly pointed out in the appended claims.

### DETAILED DESCRIPTION OF THE INVENTION

PIVANEX (pivaloyloxymethyl butyrate) is an oxyalkylene containing compound and is the pivalate ester derivative of butyric acid and is commercially available from Titan Pharmaceuticals, Inc.

In one embodiment, the present invention provides a method for enhancing antitumor activity by treatment methods using the sequential application of an oxyalkylene containing compound with certain chemotherapeutic agents. In one embodiment, the oxyalkylene containing compound is pivaloyloxymethyl butyrate.

In particular, the chemotherapeutic agent is selected from the class consisting of DNA-interactive agents, DNA-alkylating agents, tubulin-interactive agents, and platinum complexes.

In one embodiment, the therapeutic activity is effective to treat, prevent or ameliorate cancer and other proliferative disorders. The compounds of the invention in sequential administration are particularly useful for treating, preventing or ameliorating the effects of cancer and other proliferative disorders by acting as anti-proliferative or differentiating agents in subjects afflicted with such anomalies. Such disorders include but are not limited to leukemias, such as acute promyelocytic leukemia, acute myeloid leukemia, and acute myelomonocytic leukemia, other myelodysplastic syndromes, multiple myeloma such as but not limited to breast carcinomas, cervical cancers, melanomas, colon cancers, nasopharyngeal carcinoma, non-Hodgkins lymphoma (NHL), Kaposi's sarcoma, ovarian cancers, pancreatic cancers, hepatocarcinomas, prostate cancers, squamous carcinomas, other dermatologic malignancies, teratocarcinomas, T-cell lymphomas, lung tumors, gliomas, neuroblastomas, peripheral neuroectodermal tumors, rhabdomyosarcomas, and prostate tumors and other solid tumors. The oxyalkylene containing compound may have anti-proliferative effects on non-cancerous cells, and may be of use to treat benign tumors and other proliferative disorders such as psoriasis.

In another embodiment the therapeutic activity is effective to treat or ameliorate leukemia, squamous cell carcinoma and neuroblastoma.

Cancers which may be particularly effectively treated by the method of this invention include mammalian cancers, especially human cancers. Cancers that are particularly treatable by the method of this invention are cancers with sensitivity to inducers of apoptosis. Such cancers include cancers of the breast, colon and rectum, lung, liver, ovary, uterine cervix, urinary bladder, stomach, pancreas, and lymphomas, myelomas, and leukemias. Cancers particularly treatable by the method of this invention with the sequential treatment include breast, ovarian, colorectal, and non-small cell lung cancers.

In biological evaluations, pivaloyloxymethyl butyrate has been demonstrated to be a non-myeloablative and non-myelosuppressive agent. Unlike traditional cancer therapies, the oxyalkylene containing compound has been shown to induce changes in gene expression in cancer cells, causing them to undergo apoptosis. Pivaloyloxymethyl butyrate functions as a differentiating agent with activity against multiple tumor cell types in culture.

In preliminary Phase I/II clinical trials for the treatment of lung cancer and refractory malignancies metastatic to the liver, pivaloyloxymethyl butyrate appears to be well tolerated.

Testing of a pivaloyloxymethyl butyrate dose and schedule interactions with chemotherapy in tissue culture was undertaken to facilitate clinical planning.

Growth inhibitory and cytotoxic effects of an oxyalkylene containing compound with tumor cells were found to be time and concentration dependent. Cytotoxicity, which was tumor cell line dependent, was observed following a 6-hour exposure of tumor cells to pivaloyloxymethyl butyrate at concentrations greater than 125 μM and following 72 hours of treatment with pivaloyloxymethyl butyrate at concentrations greater than 25 μM.

Morphologic changes and growth inhibition of tumor cells were observed at a pivaloyloxymethyl butyrate doses as low as 5 to 25 μM following 72 hours of treatment. The observed morphologic changes consistent with a more differentiated phenotype prompted investigation of oncogene expression following exposure to pivaloyloxymethyl butyrate. A 72-hour exposure of tumor cells to pivaloyloxymethyl butyrate at doses of 10-50 μM resulted in significant suppression of p53, c-myc and ras. Altered expression of p53, c-myc and ras is associated with chemoresistance, suggesting that pretreatment with PIVANEX at doses sufficient to suppress oncogene expression but not to be directly cytotoxic could augment the action of cytotoxic drugs and thereby be additive or synergistic with current chemotherapeutics.

The synergistic effects from the pretreatment of tumor cells with pivaloyloxymethyl butyrate were demonstrated with human cancer cells. Human T24 bladder and A549 lung cancer cells were exposed to increasing concentrations of pivaloyloxymethyl butyrate for 6 or 72 hours followed by exposure to various chemotherapeutic agents. At lower doses 6-hour treatment with pivaloyloxymethyl butyrate did not enhance cytotoxicity by these agents, but 72-hour treatment of these cell lines with pivaloyloxymethyl butyrate did enhance drug-induced cytotoxicity even at concentrations of the chemotherapeutic agents that were not directly cytotoxic. At higher dosages the increase in cytotoxicity following the sequential regimen would show after a shorter induction period with pivaloyloxymethyl butyrate as discussed above.

Human Non-Small Cell Lung Carcinoma Cell Lines, H522 and NCI-H23, were evaluated for growth inhibitory activity of Pivanex, alone and in combination with the standard chemotherapeutic agent, docetaxel. Cells were treated for three days with Pivanex followed by 24 hours of exposure to docetaxel or the cells were treated for 24 hours with docetaxel followed by three days of exposure to Pivanex. The cell growth inhibition was judged at the end of the 4-day experiments by a standard staining technique using MTS tetrazolium vital stain. Cell growth was graphed vs. the various proportions of the two drugs in an isobologram analysis to determine if the sequential drug treatments were additive, synergistic or less than additive. The sequence of Pivanex followed by docetaxel yields a data pattern suggestive of an additive or synergistic interaction between these two agents. In contrast, the sequence of docetaxel followed by Pivanex yields a different pattern, one suggestive of less than additive or antagonistic effects. The results from the sequencing studies in H522 and NCI-H23 cells support the preferred sequence of initial Pivanex exposure followed by treatment with docetaxel.

Sequential Combination Therapy

The oxyalkylene containing compound is used in combination with one or more chemotherapeutic agents for the treatment of cancer or tumors. These combinations are administered sequentially, where the oxyalkylene containing compound is administered prior to the administration of the chemotherapeutic agents.

The sequential regimen provides for the administration of the oxyalkylene containing compound for an induction period, after which a chemotherapeutically effective amount of a member of the class consisting of tubulin interactors, DNA-interactive agents, DNA-alkylating agents, and platinum complexes is administered to the mammal or host cells.

The oxyalkylene containing compound may be administered for an induction period of from about more than 2, preferably more than 4, more preferably more than 6 hours to about 120 hours prior to the administration of the chemotherapeutic agents. In one embodiment of the invention, the oxyalkylene containing compound may be administered for an induction period of between about 24 hours and 96 hours prior to the administration of the chemotherapeutic agents.

In another embodiment, pivaloyloxymethyl butyrate is administered for an induction period of from about 48 to about 84 hours after the oxyalkylene containing compound. In another embodiment of the invention, pivaloyloxymethyl butyrate is administered for an induction period of from about 54 to 78 hours prior to the administration of the chemotherapeutic agent. In a further embodiment the induction period is reduced to 48, 36, 24, 12, 6, 4, and slightly more than 2 hours of Pivanex exposure.

The chemotherapeutic agents which can be used with the oxyalkylene containing compound are generally grouped as DNA-interactive or alkylating agents, tubulin-interactive agents, and platinum complexes. Each of the groups of chemotherapeutic agents can be further divided by type of activity or compound. The chemotherapeutic agents used in combination with the oxyalkylene containing compound includes certain members of these groups. For a detailed discussion of chemotherapeutic agents and their method of administration, see Dorr, et al, Cancer Chemotherapy Handbook, 2d edition, pages 15-34, Appleton & Lange (Connecticut, 1994), the disclosure of which is incorporated by reference in its entirety.

DNA-interactive agents include the alkylating agents, e.g. carboplatin, cisplatin, oxaliplatin, cyclophosphamide, dacarbazine or temozolomide, a pyrimidine-based nucleoside, such as gemcitabine or the purine-based nucleoside fludarabine. Dacarbazine is one of the drugs of choice for the treatment of multiple melanoma.

The platinum complexes include cisplatin, carboplatin and oxaliplatin.

Tubulin interactive agents act by binding to specific sites on tubulin, a protein that polymerizes to form cellular microtubules. Microtubules are critical cell structure units. When the interactive agents bind on the protein, the microtubules are stabilized or depolymerized according to type of agent used. Tubulin interactive agents include colchicine, vincristine and vinblastine, vinorelbine, paclitaxel, and docetaxel.

In one embodiment of the present invention, the oxyalkylene containing compound is administered with the chemotherapeutic agents selected from the group consisting of cisplatin, carboplatin, oxaliplatin, gemcitabine, taxol, docetaxel, and paclitaxel. In another embodiment of the present invention, the oxyalkylene compound used for administering with chemotherapeutic agents is pivaloyloxymethyl butyrate. The present invention is directed to a method of increasing therapeutic activity of certain compounds by sequentially administering therapeutic dosages of the oxyalkylene containing HDAC inhibitors to a patient or host cells and an amount of a chemotherapeutic agent effective to provide increase the therapeutic activity. In general, increasing of therapeutic activity means that the therapeutic effect of a particular compound (i.e., the oxyalkylene-containing compound and/or the chemotherapeutic agent) will be greater, increased therapeutic effectiveness, in a patient or host cells than it normally would in the absence of the chemotherapeutic combination. Increasing therapeutic activity includes enhancement of that activity. Furthermore, sequentially administering a chemotherapeutic agent with a particular compound of the invention can also cause synergistic effects and allow administration of lower doses of the chemotherapeutic agent than would be needed to achieve the same therapeutic effectiveness in the absence of the oxyalkylene containing compound.

Definitions:

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, an “oxyalkylene containing compound” is a compound having the formulas (I), (I), and (III):
X—CH₂—CHX—CHX—C(═O)—O—Z (I)
CH₃—CO—CH₂—C(═O)—O—Z (II)
CH₃—CH₂—CO—C(═O)—O—Z (III)

wherein X is H, or one X only may be OH; Z is —CHR—O—(O═)C—R′, R represents a member selected from the group consisting of hydrogen and alkyl, and R′ represents a member of the group consisting of alkyl, aminoalkyl, aralkyl, aryl, alkoxy, aralkoxy and aryloxy, in which aryl by itself, and aryl in aralkyl, aralkoxy and aryloxy, are each selected from the group consisting of sub-groups (a) and (b), wherein (a) is unsubstituted phenyl, naphthyl, furyl or thienyl, and (b) is phenyl, naphthyl, furyl or thienyl, each of which is substituted by at least one substituent selected from the group consisting of alkyl, alkoxy or halogen, provided that in (I) when X is H and R′ is propyl, then R is alkyl which contains at least three carbon atoms. The oxyalkylene containing compound includes pivaloyloxymethyl butyrate.

As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “chemotherapeutically effective amount” is meant an amount of a compound of the present invention effective to yield the desired chemotherapeutic response. For example, an amount effective to delay the growth of or to cause a cancer, either a sarcoma or lymphoma, or to shrink the cancer or prevent metastasis, or increase the survival time of a mammal. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

As used herein the terms “mg/m²” or “g/m²” in certain instances, depending on the individual chemotherapeutic agent, may refer to a daily dose or the dose administered during the course of the treatment or the treatment period or treatment cycle. In a few instances dosages are given in mg/kg/day (for example, for dacarbazine). In a number of instances the chemotherapeutic drug is administered during certain days or periods during the course of the treatment. The days or periods during which the drug is administered is often followed by resting periods during which no drug is administered. For example, a customary dose for docetaxel would be 75 mg/m² every three weeks. A typical dosage for the orally administered drug temozolomide would be 28 days in which for the first five days a total dose of 750 mg/m² would be administered, followed by a resting period of 23 days. A customary dose for pivaloyloxymethyl butyrate would be 2.5, 3, 4, 5, 6, 7 g/m² per day of treatment.

A “pharmaceutical salt” is salt of a chemotherapeutic agent which has been modified by making acid or base salts of the compounds. Examples include, but are not limited to, mineral or organic acid salts of basic residues such as amines; alkali or organic salts of acidic residues such as carboxylic acids, and the like. Pharmaceutically acceptable salts include, but are not limited to, hydro halides, sulfates, methosulfates, methanesulfates, toluenesulfonates, nitrates, phosphates, maleates, acetates, lactates and the like. Pharmaceutically-acceptable salts of the compounds of the invention can be prepared by reacting the free acid or base forms of these compounds with a stoichiometric or greater amount of the appropriate base or acid in water or in an organic solvent, or in a mixture of the two; generally, non-aqueous media like ether, ethyl acetate, ethanol, isopropanol, or acetonitrile are preferred. The salts of the invention can also be prepared by ion exchange, for example. Lists of suitable salts are found in Remington's Pharmaceutical Sciences, 17th ed., Mack Publishing Company, Easton, Pa., 1985, p. 1418 or The Merck Index, Thirteenth Edition, 2001, Published by Merck Research Laboratories Division of Merck & Co., Inc. on pages MISC-22 and MISC-23, the disclosure of which is hereby incorporated by reference in its entirety. Pharmaceutically acceptable salts also include amino acid salts such as arginine, lysine, glutamic and aspartic acid salts.

As used herein, a “pharmaceutical carrier” is a pharmaceutically acceptable solvent, suspending agent or vehicle, for delivering an oxyalkylene containing compound in conjunction with other chemotherapeutic agents to the animal or human. The carrier may be liquid or solid and is selected with the planned manner of administration in mind. Liposomes are also a pharmaceutical carrier.

As used herein, “cancer” refers to all types of cancer or neoplasm or malignant tumors found in mammals, including carcinomas and sarcomas. Examples of cancers are cancer of the brain, breast, pancreas, cervix, colon, head & neck, kidney, lung, non-small cell lung, melanoma, mesothelioma, ovary, sarcoma, stomach, uterus and Medulloblastoma.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). The P388 leukemia model is widely accepted as being predictive of in vivo anti-leukemic activity. It is believed that compound that tests positive in the P388 assay will generally exhibit some level of anti-leukemic activity in vivo regardless of the type of leukemia being treated. Accordingly, the present invention includes a method of treating leukemia, and, preferably, a method of treating acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Sarcomas which can be treated with an oxyalkylene containing compound and chemotherapeutic agent include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas which can be treated with an oxyalkylene containing compound and another chemotherapeutic agent include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas which can be treated with an oxyalkylene containing compound and a chemotherapeutic agent include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

Additional cancers which can be treated with an oxyalkylene containing compound according to the invention include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

As used herein, the “chemotherapeutic agents” include but are not limited to DNA-interacting or alkylating agents, such as certain pyrimidine or purine nucleosides, platinum-based cytotoxic drugs, and tubulin interactors. Examples of the “chemotherapeutic agents” of the invention include but are not limited to cisplatin, carboplatin, oxaliplatin, paclitaxel, docetaxel, gemcitabine, and fludarabine.

As used herein “combination therapy” or “adjunct therapy” means that the patient in need of the drug is treated or given another drug for the disease in conjunction with the oxyalkylene containing compound. This combination therapy is a sequential therapy, where the patient is treated first with the oxyalkylene containing compound, for example, pivaloyloxymethyl butyrate and then with one or more of the other chemotherapeutic drugs.

The term “induction period” means a chemotherapy treatment period during which substantially exclusively the oxyalkylene containing compound will be administered, and not the other chemotherapeutic drugs. The term “substantially exclusively” in this regard means that no more than 10% of the other chemotherapeutic drugs, preferably no more than 5%, most preferably 0% of the other chemotherapeutic drugs will be adininistered during the induction period. The induction period sensitizes the treated tumor cells to become more receptive to chemotherapy with the other chemotherapeutic drugs that are administered after the induction period. It is understood that the oxyalkylene containing HDAC inhibitor administered during the induction period is not limited to constant daily dosages but may include the administration of variable dosages of the HDAC inhibitor. Similarly, the dosages of the other chemotherapeutic agent administered after the induction period may be constant or variable. The term “variable” in this context includes all dosage variations except constant dosages. That means, for example that the dosage may be increased or decreased from Day 1 to Day 2 of the induction period or after the induction period.

The term “histones” describes highly basic polypeptides, which are classified as lysine-rich, slightly lysine-rich and arginine-rich. Many of the basic amino acids are clustered on amino-terminal tails. They are highly polycationic and interact with the polyanionic backbone of DNA to produce uncharged nucleoproteins. The histones participate in interactions essential for maintaining chromatin, the structural material of chromosomes. Histones and their functions are described in more detail in Thomas M. Devlin Textbook of Biochemistry with Clinical Correlations, Wiley-Liss, 1992, p. 637, 639-641, the disclosure of which is hereby incorporated by reference in its entirety.

The term “histone deacetylase(s) inhibitor” or “HDAC” inhibitor describes a class of molecules that block the deacetylation of histones. Acetylation of histones will lead to the neutralization of the polycationic character of the histones which leads to a partial unwrapping of the nucleosome, the basic unit structure of the chromatin. This results in a more relaxed DNA confirmation, provides access of the transcriptional apparatus to the DNA and promotes gene expression. HDAC inhibitors retain the histones in an acetylated stage and induce growth arrest and apoptosis in a variety of human cancer cells. HDAC inhibitors are described in more detail by Paul A. Marks et al. in Curr Opin Oncol 2001, 13:477-483, 2001, the disclosure of which is hereby incorporated by reference in its entirety.

All the features, characteristics and ranges described for the invention, whether in an embodiment, whether described as preferred or not, may be combined with each other. For example, a preferred feature or dosage range for the HDAC inhibitor may be combined with a more broadly defined, not preferred feature or dosage range for the second chemotherapeutic agent described herein.

The oxyalkylene containing compound in combination with one or more chemotherapeutic agents is administered by any conventional means available for use in conjunction with pharmaceuticals, either as individual therapeutic agents or in combination with other therapeutic agents. The amount and identity of a chemotherapeutic agent that is used with the oxyalkylene containing compound in treating cancer, tumor, leukemia, or other related diseases will vary according to patient response and physiology, type and severity of side effects, the disease being treated, the preferred dosing regimen, patient prognosis or other such factors.

Where the oxyalkylene containing compound is used in combination with other therapeutic agent(s), the ratio of the oxyalkylene containing compound to the other therapeutic agent will be varied as needed according to the desired therapeutic effect, the observed side-effects of the combination, or other such considerations known to those of ordinary skill in the medical arts. Generally, the ratio of the oxyalkylene containing compound to other therapeutic agent will range from about 0.5%: 99.5% to about 99.5%: 0.5% on a weight basis.

The amount of the oxyalkylene containing compound administered before the other therapeutic agents to treat cancer, tumors, or other diseases, the respective doses and the dosing regimen of the oxyalkylene containing compound and the other therapeutic agent may vary. The sequential therapy can be within a reasonable time within the range of the induction period after the completion of the first therapy before beginning the second therapy.

The dosage range of administration of PIVANEX is from about 0.01 g/m²/day to about 10 g/m²/day, that is more than 2, 3, 4, 5, 6 or 7 g/m²/ day. Often this dosage range is about 0.1 mg/m²/day to about 5 g/m²/day.

The dosage range of administration of paclitaxel in combination with an oxyalkylene containing compound is from about 10 mg/m² to about 200 mg/M² per course of the treatment. Preferably this dosage range is about 20 mg/m²/day to about 150 mg/m². The Physicians Desk Reference, 2003, pp. 2193 provides further dosage guidance (the disclosure of which is incorporated by reference in its entirety).

The dosage range of administration of gemcitabine in combination with an oxyalkylene containing compound is up to 10000 mg/m² for a treatment period of up to twelve weeks. Within this range, a dosage range of about 100 mg/m² to about 8000 mg/m² may be appropriate. The Physician's Desk Reference, 2003, ₅₇th edition, pp. 1837 (the disclosure of which is incorporated by reference in its entirety) recommends the following dosing regimen for gemcitabine: 1000 mg/m² over 30′ once weekly for up to 7 weeks, followed by one week of rest, followed by once weekly treatment for 3 consecutive weeks-out of four weeks. Gemcitabine is one of the drugs of choice for the treatment of pancreatic cancer, non-small cell lung cancer and related diseases. Administration by the intravenous route is preferred. The product is available in vials of 200 mg and 1 g of the hydrochloride salt for intravenous administration.

The dosage range for taxol would be 10 mg/m² to 500 mg/m², preferably 40 to 300 mg/m² per course of therapy.

The dosage range of administration of docetaxel in combination with an oxyalkylene containing compound is from about 10 mg/m² to 200 mg/m² per course of the treatment, preferably 50 mg/m² to 150 mg/m². More preferably this dosage range is about 60 mg/m² to about 100 mg/m². The Physician's Desk Reference, 2003, pp. 773 ( the disclosure of which is incorporated by reference in its entirety) recommends the following dosing regimen for docetaxel for the treatment of breast cancer including metastatic breast cancer: 100 mg/m² as 1 hr infusion every three weeks. The preferred treatment regimen for non-small cell lung cancer is in the range of 60 mg/m² to 100 mg/m², more preferably 75 mg/m² over 1 hour every three weeks given intravenously.

The effective amount of carboplatin administered in combination with an oxyalkylene containing compound is in a dosage range from about 1 mg/m² to about 1000 mg/m² per course of the treatment. The preferred range of carboplatin is from about 100 mg/m² to about 500 mg/m² per course of the treatment. The Physician's Desk Reference, 2003, pp. 1126 (the disclosure of which is incorporated by reference in its entirety) recommends the following dosing regimens for carboplatin: 360 mg/m² on Day 1 of every four weeks or use of the Calvert Formula as described on page 1129. The product is available in single dose vials of 50, 150 and 450 mg for intravenous infusion. Carboplatin is one of the drugs of choice for the treatment of the various forms of ovarian cancer including ovarian carcinoma and related diseases.

An effective amount of cisplatin in combination with an oxyalkylene containing compound is administered in a dosage range from about 1 mg/m² to 300 mg/m² per course of the treatment.

Oxaliplatin in combination with an oxyalkylene containing compound is administered in a dosage range from about 10 mg/m² to about 250 mg/m² per course of the treatment. The Physician's Desk Reference, 2003, pp. 2999 (the disclosure of which is incorporated by reference in its entirety) recommends the following dosing regimen for oxaliplatin: 85 mg/m² given intravenously over 120′ every two weeks. Oxaliplatin is one of the drugs of choice for the treatment of colorectal cancer or cancer of the rectum or related diseases. The product comes in vials of 50 or 100 mg.

Dacarbazine in combination with an oxyalkylene containing compound is administered in dosages of 0.5 to 10 mg/kg/day, preferably 1 to 8 mg/kg/day for 10 days. The Physician's Desk Reference, 2003, pp. 885 (the disclosure of which is incorporated by reference in its entirety) recommends the following dosing regimen for dacarbazine: 2 to 4.5 mg/kg/day for 10 consecutive days. The treatment may be repeated at four week intervals. An alternative regimen would be 250 mg/m²/day for five days, and the treatment may be repeated every three weeks. Dacarbazine is one of the drugs of choice for the treatment of metastatic malignant melanoma or Hodgkin's disease or related diseases. The drug comes in 100 mg and 200 mg vials for intravenous injection.

Temozolomide is one of the orally given chemotherapeutic drugs. In combination with an oxyalkylene containing compound it is administered in dosages of 500, 750, 1000 and 1250 mg/m² as the total dose per course of the therapy which is usually 5 days. The Physician's Desk Reference, 2003, pp. 3081 (the disclosure of which is incorporated by reference in its entirety) recommends the following dosing regimen for temozolomide: an initial dose of 150 mg/m² orally once daily for five consecutive days per 28-day treatment cycle. That dose may be increased to 200 mg/m² with platelet count monitoring. Temozolomide is one of the drugs of choice for the treatment of refractory anaplastic astrocytoma or related disorders. It comes in 250 mg capsules.

In one embodiment pivaloyloxymethyl butyrate is administered at a dosage of about 0.5 g/m²/day to 5 g/m²/day for three consecutive days followed by about 50 mg/m² to 100 mg/m² of docetaxel, on Day 4.

It is well understood that the dosage ranges indicated herein are for general guidance only. The ratio for the dosage range for the administration of the oxyalkylene containing compound with the chemotherapeutic agents may be determined from the effective dosage ranges of the oxyalkylene group containing HDAC inhibitor (for example, pivaloyloxymethyl butyrate) and the chemotherapeutics agents provided above, and may also be determined from the effectiveness of the treatment for a given dosage range and ratios. Treating physicians have significant flexibility and apply their professional judgment what regimen would work best for each individual patient. It is also understood that the sequential administration disclosed herein and the additive or more than additive (synergistic) effect of the regimen administered may require dosage changes or adjustments that may deviate from the dosage ranges disclosed herein.

The exact regimen will also depend on the disease being treated, the severity of the disease and the response to the treatment.

The identity of the chemotherapeutic agent, the pharmaceutical carrier and the amount of compound administered will vary widely depending on the species and body weight of mammal and the type of cancer being treated. The dosage administered will also vary depending upon known factors, such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

An oxyalkylene containing compound, and one or more chemotherapeutic agent preferably are administered separately in two or more different dosage forms. These can be administered independently by the same route or by two or more different routes of administration depending on the dosage forms employed.

Suitable pharmaceutical compositions and dosage forms will preferably comprise an oxyalkylene containing compound and one or more chemotherapeutic agents.

The dose and the range of chemotherapeutic agent will depend on the particular agent and the type of cancer being treated. One skilled in the art will be able to ascertain the appropriate dose.

Dosage Forms

The sequential combination may also be administered in oral, intravenous (bolus or infusion), intraperitoneal, subcutaneous, or intramuscular form, all using dosage forms well known to those of ordinary skill in the pharmaceutical arts.

An oxyalkylene containing compound in combination with one or more chemotherapeutic agents is typically administered in admixture with suitable pharmaceutical diluents, extenders, excipients, or carriers (collectively referred to herein as a pharmaceutically acceptable carrier or carrier materials) suitably selected with respect to the intended form of administration and as consistent with conventional pharmaceutical practices. The unit will be in a form suitable for oral, rectal, intravenous injection or parenteral administration. In addition, topical and other modes of administration including intraurethral, intravaginal, or intrabladder may be useable

An oxyalkylene containing compound in combination with one or more chemotherapeutic agents can be administered alone but is generally mixed with a pharmaceutically acceptable carrier. This carrier can be a solid or liquid, and the type of carrier is generally chosen based on the type of administration being used.

Specific examples of pharmaceutical acceptable carriers and excipients that may be used as described in Remington: The Science and Practice of Pharmacy, A. Gennaro, ed., 20th edition, Lippincott, Williams & Wilkins, Philadelphia, Pa.; Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995). Pivanex is administered intravenously (IV) as an emulsion in Intralipid 20% IV Fat Emulsion with 200-proof ethanol. It is diluted in ethanol (2 mL/g of Pivanex) and then added to Intralipid 20% IV Fat Emulsion to produce a stock emulsion of a 20 mg Pivanex/mL.

The oxyalkylene containing compound in combination with one or more chemotherapeutic agents can also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines.

A sustained release dosage form may also be formulated which first releases Pivanex over the induction period, followed by the release of an orally active chemotherapeutic agent such as temozolomide. The preparation of such dosage forms is known in the art.

In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington: The Science and Practice of Pharmacy, a standard reference text in this field.

Parenteral and intravenous forms may also include minerals and other materials to make them compatible with the type of injection or delivery system chosen.

Useful pharmaceutical dosage forms for administration of an oxyalkylene containing compound in combination with one or more chemotherapeutic agents are illustrated as follows:

Injectable Solution

A parenteral composition suitable for administration by injection is prepared by stirring 1.5% by weight of active ingredients in 10% by volume propylene glycol and water. The solution is made isotonic with sodium chloride and sterilized.

Method of Treatment

The method of treatment can be any suitable method which is effective in the treatment of the particular cancer or tumor type being treated. Treatment may be, rectal, parenteral or intravenous administration or by injection into the tumor or cancer. The method of administering an effective amount also varies depending on the disorder or disease being treated. In one embodiment of the present invention, parenteral treatment by intravenous, subcutaneous, or intramuscular application of the oxyalkylene containing compound in combination with one or more chemotherapeutic agents, formulated with an appropriate carrier, additional cancer inhibiting compound or compounds or diluent to facilitate application will be the preferred method of administering the compounds to warm blooded animals.

One skilled in the art will recognize that the efficacy of the oxyalkylene containing compound in combination with one or more chemotherapeutic agents can be ascertained through routine screening using known cancer cell lines both in vitro and in vivo. Cell lines are available from American Tissue Type Culture or other laboratories.

The following examples are illustrative and not intended to be limiting of the invention.

### EXAMPLE 1

Tumor Cell Lines and Cell Cultures:

T24 Bladder transitional carcinoma cells, Calu-6 anaplastic lung carcinoma cells, and SK-MES-1 squamous lung cell carcinoma cell cultures were purchased from the American Type Culture Collection (Manassas, Va.). All cell lines were cultured in RPMI-1640 with 10% fetal calf serum. For culture in microtiter dishes, cells were plated at a density of 10⁴ cells/well and incubated at 37° C., 5% CO₂ overnight.

After overnight incubation, drugs were added to wells in quadruplicate and incubated for 6 to 96 hours in the presence or absence of the oxyalkylene containing compound. Before the media was removed, fresh media were added with cytotoxic drug, and the cultures were incubated for another 6 hours.

The wells were then washed and medium without drug was added to each well. Microtiter plates were subsequently incubated for an additional 3 days before cell density was determined by a Crystal Violet Assay. Some cultures were alternatively fixed in methanol and stained with hematoxylin and eosin (H&E) stain for 5 minutes prior to microphotography.

Antitumor Drugs:

PIVANEX (pivaloyloxymethyl butyrate) was obtained from Titan Pharmaceuticals Inc. (South San Francisco, Calif.) and was admixed with 0.05% Intralipid™ (Fresenius Kabi, Clayton, N.C.) prior to addition to culture media. The cytotoxic drugs gemcitabine (Gemzar; Eli Lilly and Co., Indianapolis, Ind.), cisplatin (Platinol, Bristol-Myers Squibb Co., Princeton, N.J.), paclitaxel (Taxol; Bristol-Myers Squibb Co., Princeton, N.J.), and docetaxel (Taxotere, Aventis Pharmaceuticals Products, Inc., Bridgewater, N.J.) were studied with PIVANEX.

Crystal Violet Microtiter Assay:

Cells in microtiter wells were rinsed with PBS and fixed in two volumes of 100% cold methanol for 5 minutes. One volume of 0.5% crystal violet in 20% methanol was added and the attached cells stained for 10 minutes. The cells were then washed and crystal violet was eluted with 0.1 N sodium citrate buffer and read on an ELISA plate reader at 490 nm.

Oncogene Expression Analysis:

Tumor cell culture mRNA was isolated by standard methods, and RT-PCR was conducted to determine levels of c-Myc oncogene expression. The c-Myc primer pair used for RT-PCR cycling was:

| | |
|---|---|
| c-Myc: | 5′-GCC AAG CCA GTT CCA TTA AA-3′ |
| | 3′-ACT CCC GGA CTG TCT GTCAT-5′ |

The c-Myc primer pair was cycled by the following program: 95° C. for 3 min., then 30 cycles of (95° C. for 1 min., then 55° C. for 1 min., then 72° C. for 1 min.) followed by holding temperature at 72° C. for 7 min. before ramping temperature to 4° C. Samples were analyzed quantitatively by 1% agarose gel electrophoresis and gel densitometry.

TABLE 1^{a}
Pivanex Decreases Expression of c-Myc Oncogene

| Pivanex | Calu-6 | T24 Bladder | | T24 |
|---|---|---|---|---|
| conc μM | NSCLC | Cancer | Calu-6 | Bladder |
| 96 hour | PCR units | PCR units | NSCLC | Cancer |
| exposure | cells n = 2 | cells = 3 | SD | SD |
| 0 | 880 | 706 | 66 | 30 |
| 10 | 893 | 760 | 20 | 59 |
| 25 | 743 | 972 | 137 | 234 |
| 50 | 711 | 966 | 0 | 72 |
| 100 | 647 | 640 | 20 | 82 |
| 200 | 460 | 560 | 1 | 31 |

^{a}Table plotting the PIVANEX concentration in μM (at 96 hour exposure) against the arbitrary PCR unit, showing that PIVANEX decreases the expression of c-Myc oncogene.

TABLE 2^{a}
Cytotoxicity of Pivanex and Cisplatin is Additive in T24 Bladder Cancer Cell Line

| Cisplatin | 0 μM | 10 μM | 50 μM | 100 μM | 0 μM Piv | 10 μM Piv | 50 μM | 100 μM Piv |
|---|---|---|---|---|---|---|---|---|
| dose (μM) | Pivanex | Pivanex | Pivanex | Pivanex | SD | SD | Piv SD | SD |
| 0 | 0.479 | 0.48 | 0.422 | | 0.012 | 0.02 | 0.047 | |
| 2.5 | 0.486 | 0.481 | 0.406 | 0.424 | 0.023 | 0.009 | 0.017 | 0.006 |
| 5 | 0.434 | 0.427 | 0.336 | 0.418 | 0.021 | 0.022 | 0.03 | 0.046 |
| 10 | 0.404 | 0.378 | 0.318 | 0.31 | 0.012 | 0.028 | 0.034 | 0.027 |
| 20 | 0.25 | 0.287 | 0.231 | 0.218 | 0.012 | 0.036 | 0.034 | 0.003 |

^{a}Table plotting the PIVANEX concentration in μM treatment and cisplatin concentration (in μM) after PIVANEX treatment against T24 cancer cell viability (A⁴⁹⁰ Absorbance), showing cytotoxicity of PIVANEX and cisplatin is additive.

TABLE 3^{a}
Pivanex Overcomes Resistance to Paclitaxel SK-MES-1
Non-Small Cell Lung Cancer Cells

| Paclitaxel | 0 μM | 10 μM | 50 μM | 0 μM | 10 μM Piv | 50 uM |
|---|---|---|---|---|---|---|
| conc. | Pivanex | Pivanex | Pivanex | Piv SD | SD | Piv SD |
| 0 | 0.737 | 0.743 | 0.769 | 0.016 | 0.089 | 0.051 |
| 25 | 0.655 | 0.669 | 0.682 | 0.037 | 0.02 | 0.016 |
| 50 | 0.627 | 0.581 | 0.559 | 0.016 | 0.051 | 0.037 |
| 100 | 0.42 | 0.118 | 0.217 | 0.113 | 0.024 | 0.019 |

^{a}Table plotting the PIVANEX concentration in μM treatment and paclitaxel concentration (in μM) after PIVANEX treatment against SK-MES-1 cancer cell viability (A⁴⁹⁰ Absorbance), showing PIVANEX overcomes resistance to paclitaxel.

TABLE 4^{a}
Cytotoxicity of Pivanex and Cisplatin is Additive
in Calu-6 Non-Small Cell Lung Cancer Cells

| | | 0 uM | 2.5 μM | 5 μM | 10 uM | 20 μM |
|---|---|---|---|---|---|---|
| uM Pivanex | | cisplatin | cisplatin | cisplatin | cisplatin | cisplatin |
| 0 | uM Pivanex | 0.666 | 0.608 | 0.557 | 0.328 | 0.238 |
| 10 | uM Pivanex | 0.617 | 0.58 | 0.527 | 0.368 | 0.246 |
| 50 | uM Pivanex | 0.577 | 0.486 | 0.443 | 0.365 | 0.242 |
| 100 | uM Pivanex | 0.284 | 0.233 | 0.16 | 0.115 | 0.081 |

^{a}Table plotting the PIVANEX concentration in μM treatment and cisplatin concentration (in μM) after PIVANEX treatment against Calu-6 cancer cell viability (A⁴⁹⁰ Absorbance), showing cytotoxicity of PIVANEX and cisplatin is additive.

TABLE 5^{a}
Cytotoxicity of Pivanex and Cisplatin is Additive
in Calu-6 Non-Small Cell Lung Cancer Cells

| | | 0 μM | 2.5 μM | 5 μM | 10 μM | 20 μM |
|---|---|---|---|---|---|---|
| μM Pivanex | | cis SD | cis SD | cis SD | cis SD | cis SD |
| 0 | μM Pivanex | 0.026 | 0.031 | 0.041 | 0.015 | 0.009 |
| 10 | μM Pivanex | 0.038 | 0.021 | 0.075 | 0.093 | 0.043 |
| 50 | μM Pivanex | 0.011 | 0.008 | 0.016 | 0.047 | 0.023 |
| 100 | μM Pivanex | 0.009 | 0.008 | 0.057 | 0.056 | 0.013 |

^{a}SD Table plotting the PIVANEX concentration in μM treatment and cisplatin concentration (in μM) after PIVANEX treatment against Calu-6 cancer cell viability (A⁴⁹⁰ Absorbance) showing cytotoxicity of PIVANEX and cisplatin is additive.

TABLE 6^{a}
Cytotoxicity of Pivanex and Gemcitabine (“Gem”) is Additive in Calu-6 Non-
Small Cell Lung Cancer Cells

| | | | | | | | 0 nM | 25 nM | 50 nM | 100 nM | 200 nM |
|---|---|---|---|---|---|---|---|---|---|---|---|
| | | 0 nM | 25 nM | 50 nM | 100 nM | 200 nM | Gem | Gem | Gem | Gem | Gem |
| | | Gem. | Gem. | Gem. | Gem. | Gem. | SD | SD | SD | SD | SD |
| 0 | μM Pivanex | 0.666 | 0.587 | 0.466 | 0.456 | 0.403 | 0.026 | 0.028 | 0.027 | 0.025 | 0.011 |
| 10 | μM Pivanex | 0.617 | 0.565 | 0.479 | 0.437 | 0.313 | 0.038 | 0.058 | 0.041 | 0.039 | 0.038 |
| 50 | μM Pivanex | 0.577 | 0.481 | 0.453 | 0.368 | 0.268 | 0.011 | 0.039 | 0.03 | 0.028 | 0.049 |
| 100 | μM Pivanex | 0.284 | 0.217 | 0.211 | 0.194 | 0.204 | 0.009 | 0.023 | 0.016 | 0.052 | 0.03 |

^{a}Table plotting the PIVANEX concentration in μM treatment and gemcitabine concentration (in nM) after PIVANEX treatment against Calu-6 cancer cell viability (A⁴⁹⁰ Absorbance), showing cytotoxicity of PIVANEX and gemcitabine is additive.

TABLE 7^{a}
Cytotoxicity of Pivanex and Gemcitabine is Greater than Additive in T24
Bladder Cancer Cells

| M | 0 nM | 10 nM | 50 nM | 100 nM | 0 uM | 10 uM | 50 uM |
|---|---|---|---|---|---|---|---|
| Pivanex | gemcitabine | gemcitabine | gemcitabine | gemcitabine | SD | SD | SD |
| 0 | 0.479 | 0.48 | 0.422 | | 0.01 | 0.02 | 0.052 |
| 25 | 0.479 | 0.476 | 0.409 | 0.424 | 0.023 | 0.009 | 0.017 |
| 50 | 0.455 | 0.461 | 0.418 | 0.418 | 0.015 | 0.022 | 0.03 |
| 100 | 0.414 | 0.41 | 0.401 | 0.31 | 0.012 | 0.028 | 0.034 |
| 200 | 0.41 | 0.359 | 0.366 | 0.218 | 0.012 | 0.036 | 0.034 |

^{a}Table plotting the PIVANEX concentration in μM treatment and gemcitabine concentration (in nM) after PIVANEX treatment against T24 cancer cell viability (A⁴⁹⁰ Absorbance), showing cytotoxicity of PIVANEX and gemcitabine is greater than additive.

TABLE 8^{a}
Cytotoxicity of Pivanex and Cisplatin in Greater than Additive
in SK-MES-1 Non-Small Cell Lung Cancer Cells

| | | | | | | Piv + |
|---|---|---|---|---|---|---|
| | Piv | Piv + 10 | Piv + 20 | Piv | Piv + 10 | 20 |
| Pivanex | Alone | μM | μM | Alone | μM | μM |
| conc | (Abs) | Cisplatin | Cisplatin | SD | cis SD | cis SD |
| 0 | 2.232 | 1.562 | 1.477 | 0.048 | 0.166 | 0.242 |
| 25 | 1.852 | 0.751 | | 0.11 | 0.051 | |
| 100 | 1.908 | 0.755 | | 0.127 | 0.085 | |
| 200 | 1.952 | 0.779 | | 0.02 | 0.061 | |

^{a}Table plotting the PIVANEX concentration (in μM) and cisplatin (in μM) with PIVANEX concentration (in μM) against SK-MES-1 non-small cancer cell viability (A⁴⁹⁰ Absorbance), showing cytotoxicity of PIVANEX and cisplatin is greater than additive.

TABLE 9^{a}
Cytotoxicity of Pivanex and Docetaxel is Greater than Additive
in Calu-6 Non-Small Cell Lung Cancer Cells

| | 0 μM | 10 μM | 50 μM | 0 μM | 10 μM | 50 μM |
|---|---|---|---|---|---|---|
| Docetaxel conc | Pivanex | Pivanex | Pivanex | Piv SD | Piv SD | Piv SD |
| 0 | 0.716 | 0.732 | 0.553 | 0.017 | 0.04 | 0.083 |
| 25 | 0.219 | 0.214 | 0.117 | 0.047 | 0.047 | 0.027 |
| 50 | 0.183 | 0.148 | 0.106 | 0.078 | 0.072 | 0.026 |
| 100 | 0.174 | 0.131 | 0.099 | 0.079 | 0.028 | 0.017 |
| 200 | 0.148 | 0.113 | 0.092 | 0.052 | 0.018 | 0.007 |

^{a}Table plotting the PIVANEX concentration (in μM) treatment and docetaxel concentration (in nM) after PIVANEX treatment against Calu-6 cancer cell viability (A⁴⁹⁰ Absorbance), showing cytotoxicity for PIVANEX and docetaxel is greater than additive.

The following is a summary of the effects of PIVANEX and chemotherapeutic agents on tumor cell growth:

PIVANEX was found to be additive in combination with cisplatin and greater than additive in combination with gemcitabine for the bladder cancer cell line (T24).

PIVANEX was found to be greater than additive with cisplatin or paclitaxel in chemoresistant non-small-cell lung cancer cell line (SK-MES-1).

PIVANEX was found to be additive with cisplatin, the nucleoside analog gemcitabine, and greater than additive with the taxane docetaxel in non-small-cell lung cancer cell line (Calu-6).

For certain indications, the combination of PIVANEX with the chemotherapeutic agents was found to be additive, and for other indications, the combination had greater than additive activities.

The exposure of several tumor cell lines (Calu-6, SK-MES-1, T-24) to PIVANEX for 96 hours in culture at concentrations as low as 10-50 μM decreased the expression of c-Myc oncogene.

PIVANEX modulation of oncogene expression was time dependent as well as tumor cell-line dependent.

### EXAMPLE 2

This example shows the cytotoxicity of PIVANEX and Docetaxel in drug combination with an induction period of 3 day (72 hours) followed by 24 hours docetaxel exposure.

Methodology

Cell Lines

The human NSCLC cell lines H522 (fast growing, t ½=28 h) and H23 (slow growing; t½=38 h) purchased from ATCC (Rockville, Md.), were maintained in 75-cm 2 plastic tissue culture flasks in RPMI medium (RPMI; Nova Tech, Grand Island, N.Y.) containing 10% fetal bovine serum (FBS; Nova Tech). The cells were incubated at 37° C. in a humidified atmosphere containing 5% CO₂.

Treatments

Pivanex (Titan Pharmaceuticals) was diluted in 100% ethanol to a 1 M stock solution with a final ethanol concentration of <0.5%. Docetaxel (Taxotere TM) was generously provided by Aventis (Strasbourg, France) and dissolved in 100% dimethyl sulfoxide (DMSO) to a 1000× stock solution. For cell culture studies Pivanex and docetaxel were diluted in cell culture medium to a final concentration of <0.5% ethanol and <0.1% DMSO, respectively.

Growth Inhibition Assay

Exponentially growing cells were harvested with trypsin (0.05%)/EDTA (0.02%) and re-suspended in fresh medium containing 10% FBS. Cell suspensions in 100 μl growth medium were plated on Day 0 in 96-well microtiter plates (Falcon, Oxnard, Calif.) at a concentration of 10⁴ cells/well. The cells were incubated 24 hours at 37° C. in a humidified atmosphere containing 5% CO₂ prior to drug treatment. On Day 1, 100 μl aliquots of medium containing serially diluted concentrations of drug and vehicle were added to the cell plates and incubated for the time specified for each respective sequence. After incubation at 37° C. in a humidified incubator (5% CO₂/95% HEPA filtered air) for 4 days, 100 μl of the growth medium were removed. Cells were then incubated after the addition of 20 μl MTS tetrazolium [3-(4,5-dimethylthiazol-2-yl)-5-(3-carboxymethoxyphenyl)-2-(4-sulfophenyl)-2-tetrazolium-inner salt] compound (1.9 mg/ml in PBS, pH 6.0), for 1 h at 37° C. The MTS tetrazolium compound was bioreduced by viable cells into a colored formazan product that is soluble in cell culture medium. Absorbance was documented on a Dynex HD microplate reader at a wavelength of 490 nm. IC₅₀ values were determined by using the Prism® GraphPad software from 3 different tests, each of which involved 4 replicates for each dose determination. The data are presented as percent growth inhibition, where 0% represents the mean value in wells to which only vehicle (0.1% DMSO) was added and was calculated as follows:
% Growth Inhibition=(1−(ODₜₑₛₜ/OD_{vehicle}))×100
where ODₜₑₛₜ is the optical density of the tested sample, OD vehicle is the optical density of the vehicle in which each respected drug is dissolved.
H23 Cells Plus Pivanex/Docetaxel

In the sequencing combination studies of Pivanex followed by docetaxel, the results indicate a synergistic/additive effect for the two agents on the inhibition of tumor cell growth.

H23—Treatment with Pivanex followed by docetaxel resulted in synergistic growth inhibitory activity, whereas PIVANEX and docetaxel alone show a steep increase in H23 cell growth after 24 hour exposure.

H522—Treatment with Pivanex followed by docetaxel indicated additive/synergistic growth inhibitory activity, whereas PIVANEX and docetaxel alone show a steep increase in H522 cell growth after 24 hour exposure.

In this assay Pivanex administered to H23 cells produces within the range of Pivanex/docetaxil concentrations (expressed as a percentage of the individual agent's IC₅₀) between 60/40 to 25/75 a more than additive (synergistic) growth inhibitory effect between 5 to 25% (5% at 25/75; 12% at 50/50 and 25% at 40/60) above the additive base line. Relative to the additive base line values this effect corresponds to about 12.5%, 25% and 60% increases in growth inhibitory activity.

### EXAMPLE 3

Intralipid Formulation Procedure for Pivanex

Aseptic procedures are used, under a laminar flow hood. The appropriate amount of Pivanex is placed in a sterile vial and 2 ml of ethyl alcohol (200 proof), per gram of Pivanex, are added to the vial. The appropriate amount of Intralipid (20%) is then added to the vial to produce a stock emulsion of a 20 mg/mL concentration of Pivanex. The vial is then gently inverted several times. This emulsion is further diluted with Intralipid (20%) to produce the desired concentration.

Vehicle control

The appropriate amount of Intralipid (20%) is placed in a sterile glass vial. The appropriate amount of ethyl alcohol (200 proof) is added to produce a 4% concentration. The vial is gently inverted several times.

Although the invention has been described herein with reference to specific embodiments, conceivably, many modifications and variations therein will readily occur to those skilled in the art. The preferred embodiments are disclosed and described in detail are as exemplary and is therefore not intended to be limiting of the invention. Accordingly, all such variations and modifications are included within the intended scope of the invention.

The foregoing disclosure includes all the information deemed essential to enable those skilled in the art to practice the claimed invention. Because the cited patents or publications may provide further useful information these cited materials are hereby incorporated by reference in their entirety.

1. A method of increasing the anti-tumor activity of an inhibitor of histone deacetylase(s) (HDAC) which comprises sequential administration to a mammal or host cells of a chemotherapeutically effective amount of an oxyalkylene containing HDAC inhibitor for an induction period after which a chemotherapeutically effective amount of a member of the class consisting of tubulin interactors, DNA-interactive agents, DNA-alkylating agents, and platinum complexes is administered to the mammal or host cells.

2. The method of claim 1 where the oxyalkylene containing HDAC inhibitor is a compound having the formulas (I), (II), and (III):
X—CH₂—CHX—CHX—C(═O)—O—Z (I)
CH₃—CO—CH₂—C(═O)—O—Z (II)
CH₃—CH₂—CO—C(═O)—O—Z (III)
wherein X is H, or one X only may be OH; Z is —CHR—O—(O═)C—R′, R represents a member selected from the group consisting of hydrogen and alkyl, and R′ represents a member of the group consisting of alkyl, aminoalkyl, aralkyl, aryl, alkoxy, aralkoxy and aryloxy, in which aryl by itself, and aryl in aralkyl, aralkoxy and aryloxy, are each selected from the group consisting of sub-groups (a) and (b), wherein (a) is unsubstituted phenyl, naphthyl, furyl or thienyl, and (b) is phenyl, naphthyl, furyl or thienyl, each of which is substituted by at least one substituent selected from the group consisting of alkyl, alkoxy or halogen, provided that in (I) when X is H and R′ is propyl, then R is alkyl which contains at least three carbon atoms.

3. The method of claim 1 wherein the oxyalkylene containing compound is pivaloyloxymethyl butyrate.

4. The method of claim 1 wherein the tubulin interactor is taxol, paclitaxel or docetaxel, the DNA-interactive agent is a pyrimidine-based nucleoside analog or fludarabine, the DNA-alkylating agent is dacarbazine, temozolomide or cyclophosphamide and the platinum complex is cisplatin, carboplatin or oxaliplatin.

5. The method of claim 4 wherein the pyrimidine-based nucleoside analog is gemcitabine.

6. The method of claim 1 wherein the induction period is from about more than 2 to about 120 hours.

7. The method of claim 6 wherein the induction period is about 24 to 96 hours.

8. The method of claim 7 wherein the induction period is about 48 to 84 hours.

9. The method of claim 8 wherein the induction period is about 54 to 78 hours.

10. The method of claim 1 wherein the mammal is human.

11. The method of claim 1 wherein the effective amount of the oxyalkylene containing compound, in combination with a chemotherapeutic agent, is administered in a dosage range from about 0.01 g/m²/day to about 10 g/m²/day.

12. The method of claim 5, wherein the effective amount of gemcitabine in combination with an oxyalkylene containing compound, is administered in a dosage range from about up to 10000 for a treatment period up to twelve weeks.

13. The method of claim 4, wherein the effective amount of paclitaxel or docetaxel in combination with an oxyalkylene containing compound, is administered in a dosage range from about 10 mg/m² to about 200 mg/m² per course of the treatment.

14. The method of claim 13, wherein docetaxil is administered in a dosage between 10 mg/m² to 200 mg/m².

15. The method of claim 4 wherein the effective amount of carboplatin in combination with an oxyalkylene containing compound, is administered in a dosage range from about 10 mg/m² to about 1000 mg/m² per course of the treatment.

16. The method of claim 15 wherein the effective amount of carboplatin is administered in a dosage range from about 100 mg/m² to 500 mg/m².

17. The method of claim 4 wherein the effective amount of oxaliplatin in combination with an oxyalkylene containing compound, is administered in a dosage range from about 10 mg/m² to about 250 mg/m² per course of the treatment.

18. The method of claim 4 wherein the effective amount of cisplatin in combination with an oxyalkylene containing compound, is administered in a dosage range from about 1 mg/m² to 300 mg/m² per course of the treatment.

19. The method of claim 4 wherein the effective amount of dacarbazine is in combination with an oxyalkylene containing compound, is administered in a dosage range from about 0.5 to 10 mg/kg/day for a course of the treatment often days.

20. The method of claim 4 wherein the effective amount of temozolomide is administered in dosages of 500 to 1250 mg/m² per course of the therapy.

21. The method of claim 3 wherein pivaloyloxymethyl butyrate is administered at a dosage of about 0.5 g/m²/day to 5 g/m²/day for three consecutive days followed by about 50 mg/m² to 100 mg/m² of docetaxel on Day 4.

22. The use of an HDAC inhibitor in the manufacture of a chemotherapeutic preparation for increasing the anti-tumor activity of said HDAC inhibitor which includes the use of a chemotherapeutic agent of the class consisting of tubulin interactors, DNA-interactive agents, DNA-alkylating agents, and platinum complexes, said preparation being adapted for an induction period during which the HDAC inhibitor is administered, followed by administration of said chemotherapeutic agent.


SEQUENCE LISTING (2 sequences)

SEQ ID NO: 1 - DNA, 20 bp, Artificial Sequence
  primer
gccaagccagttccattaaa

SEQ ID NO: 2 - DNA, 20 bp, Artificial Sequence
  primer
actcccggactgtctgtcat